(19) United States
(12) Patent Application Publication
(10) Pub. No.: US 2018/0052955 A1
(43) Pub. Date: Feb. 22, 2018

(54) Health Condition Determination System

(71) Applicants: David R. Hall, Provo, UT (US); Dan Allen, Springville, UT (US); Jared Reynolds, Pleasant Grove, UT (US); Joshua Larsen, Spanish Fork, UT (US); Ben Swenson, Lehi, UT (US); Huijae Kim, Provo, UT (US); Eric Magleby, Provo, UT (US)

(72) Inventors: David R. Hall, Provo, UT (US); Dan Allen, Springville, UT (US); Jared Reynolds, Pleasant Grove, UT (US); Joshua Larsen, Spanish Fork, UT (US); Ben Swenson, Lehi, UT (US); Huijae Kim, Provo, UT (US); Eric Magleby, Provo, UT (US)

(21) Appl. No.: 15/242,929

(22) Filed: Aug. 22, 2016

(51) Int. Cl. G06F 19/00; G01N 33/49; G06F 21/62

(52) U.S. Cl. CPC G06F 19/322; G06F 21/6245; G01N 33/49

(57) ABSTRACT

A health condition determination method is disclosed. Analyte samples may be obtained from a medical toilet in individual sessions on a daily basis and analyzed over weeks, months and years. Data collected from these samples is analyzed according to a predetermined target variance. The data is filtered and recursively analyzed inputting additional sample data until the refined measured variance is within the target variance. The determined health condition is then communicated to a health professional.

### BACKGROUND

### Field of the Invention

This invention relates to systems for determining a health condition.

### Background of the Invention

Medical devices typically take a measurement and report a result. FDA approval of the instrument for certain types of measurements is usually contingent on the measurement results being within an expected error bound. In many cases the requirement for a measurement to meet a certain level of accuracy is based on a current industry standard of performance. Typical medical devices are for reporting to physicians or other health care professionals to inform decisions about diagnosis and treatment. These are time-sensitive decisions and multiple measurements over a longer period of time to improve accuracy are a luxury seldom afforded.

However, a different use of medical measurement technology exists: home monitoring. The goal in home monitoring is often to establish and monitor trends of longer term such as weeks to months or even years. Ubiquitous measurement requires a more affordable measurement technique than a one-time on-demand medical measurement and it has a somewhat orthogonal value proposition. Doctors cannot readily measure and record patient health trends, however that information is key to informing a variety of decisions, from medicine dosing to drug compliance (e.g. is the user regularly taking their medication or not?), early diagnosis and preventative medicine. Trending measurements provide additional value to users who are trying to modify behavior to achieve certain health goals such as losing weight, managing stress, hypertension, cardiovascular health, and so on.

A ubiquitous, for instance at-home, measurement (and often clinical measurements) is susceptible to a variety of stochastic influences both internal and external to the body, as well as the potential for instrumental measurement accuracy limitations potentially stemming from cost constraints. Thus, it would be beneficial to have a device that while reporting information useful to a user, can self-adjust its data averaging interval to achieve a required level of accuracy.

In summary, a key advantage posited by ubiquitous measurement is repeated measurements in a non-clinical setting. A key challenge is achieving medically-relevant accuracy. In light of the foregoing, what is needed is a new solution with self-adjustment to take advantage of ubiquitous measurement for health data trending applications while meeting the required or expected level of assurance.

### SUMMARY

This invention has been developed in response to the present state of the art and, in particular, in response to the problems and needs in the art that have not yet been fully solved by currently available systems and methods. Accordingly, an improved health measurement reporting system has been developed. Features and advantages of different embodiments of the invention will become more fully apparent from the following description and appended claims, or may be learned by practice of the invention as set forth hereinafter.

Consistent with the foregoing, a health measurement trend reporting system is disclosed. A processor executing a filtering function is disclosed. In one embodiment, the filtering function comprises a calculation step and a dynamic averaging duration. In another embodiment, the filtering function additionally comprises at least one outlier filters. A means whereby the filtering function may determine a filtered output is disclosed. A health report is disclosed which uses the filtered output from the filtering function. A medical toilet is also disclosed which may use the health measurement trend reporting system to create the health report.

Analyte Sample is defined as the health condition of the specific body part, component or system being measured. For example, the properties of the heart can be measured—analyte samples of the heart may be heart rate (in beats per minute) and blood pressure (systolic and diastolic). Another example is the property of specific gravity (SG) of urine, which can be measured from an analyte sample. Other examples include urine, vomit, feces, body excretions, pulse, blood flow, and oxygen concentration.

Health Condition is defined as the current state of health of a mammal. In general terms this refers to the overall health of an individual. Individual body parts, systems or components can be measured and analyzed to determine whether or not the individual's health is within normal ranges or parameters.

Toilet is defined as anything that receives biological waste, i.e. a receptacle, bowl, or urinal.

Health Measurement Device is defined as one of any measurement device from the group of devices, systems or methods which gather analog or digital data relating to the health of an individual. This includes sensors, detectors, analysis methods including reagent methods, chemiluminescence methods, chromatography, dynamic light scattering, imaging particle analysis, aerosol mass spectrometry, or microscope counting.

Individual is defined as either a person or an animal.

### DETAILED DESCRIPTION OF CERTAIN EMBODIMENTS

It will be readily understood that the components of the present invention, as generally described and illustrated in the Figures herein, may be arranged and designed in a wide variety of different configurations. Thus, the following more detailed description of the embodiments of the invention is not intended to limit the scope of the invention, as claimed, but is merely representative of certain examples of presently contemplated embodiments in accordance with the invention. The presently described embodiments will be best understood by reference to the claims and drawings.

Certain embodiments of the invented health measurement method include: providing an analyte sample, providing a predetermined target variance for one or more properties of the analyte sample, providing a toilet comprising one or more health measurement devices in communication with the analyte sample, providing multiple toilet-based health measurement sessions gathering data related to the analyte sample using the one or more health measurement devices, providing one or more processors connected to the one or more health measurement devices, the one or more processors receiving the data and executing a filtering function for refining the data, using the processors to track the filtered data over the multiple sessions, using the filtered data recursively to refine a measured variance for the one or more properties of the analyte sample until a refined measured variance is within the predetermined target variance, using the one or more properties of the analyte sample when the predetermined target variance is reached to determine and report a health condition related to the analyte sample.

The described health measurement method may include an analyte sample within a range of normal health conditions, or may include a sample outside a range of normal health conditions. The predetermined target variance is initially a specific predefined variance based on the analyte sample being sampled. This initial target variance is changed based on a modified standard deviation which is revised according to the filtered data after each toilet-based health measurement session specific to an individual.

The one or more health measurement devices that is in communication with the analyte sample by means of a sensor is selected from one or more of: a light based sensor, a sound sensor, a color sensor, a digital sensor, an analog sensor, a spectrometer, a refractometer, a camera based particle sizer, an x-ray, a doppler sensor, an infrared sensor, a monochromator, a sonogram sensor, a magnetic resonance imaging sensor, a cardiograph sensor, an electrocardiograph sensor, an echocardiogram sensor, a scale, a pressure sensor, a thermometer, a temperature sensor, a glucose monitor, an interferometer, a colorimeter, a stethoscope, a glucose polarization analyzer, an infrared spectroscopy device, an absorption detector, a reflectance detector, a transmission detector, a conductivity sensor, a polarographic flow analyzer, an oxygen electrode, a body fat measuring apparatus, a current or voltage electrode, a blood pressure measuring apparatus, a camera, a microscope, a particle size analyzer, an optical detector, a proximity sensor, an ultrasonic sensor, a flow sensor, a chemoreceptor, a biosensor, or an oximetry device.

These one or more health measurement devices in communication with the analyte sample may be an adjunct to the toilet. The adjunct to the toilet may be a urine hat. The toilet-based health measurement sessions may include urinating or defecating in an adjunct to the toilet.

The analyte sample may be one or more of: glucose, blood pressure, red and white blood cells, heart rate, lung sounds, body temperature, cholesterol, bone marrow, antigens, cancer, minerals, vitamins, nutrients, proteins, hormones, hCG levels, body weight, toxic substances, drugs, waste substances, urea, or lactic acid.

The database may include a medical database accessible by an individual or an individual's chosen medical personnel via a computer or phone with an internet connection. The health data may only be recorded, stored and be accessible locally. The database may be a national medical database. Tracking of the data may occur over an extended period of time. This period of time may include a segment of reporting including multiple daily sessions throughout one day up to 15 days. Another segment may include 1-30 days, an additional segment may include 1-90 days, or may include 1 day up to a year.

The data related to each individual is kept separate in the database from all other individuals. Multiple sessions over an extended period of time are kept in that specific individual's file.

Combined data from all individuals is also kept in a combined file for analysis of trends in the general population of individuals that may inform the filtering and refinement of the analyte sampling of each individual. Global or regional trends are identified and utilized to inform which samples to take and how to refine this data based on the known issues.

The toilet-based health measurement sessions may include urinating or defecating in the toilet. The data gathered relating to the analyte sample may occur every time the toilet is used. The health data measurements may be taken while a person is using the toilet. The specific health condition reports for each individual are kept separate in the database from the reports of other individuals.

This medical device takes data until it is sure the measurement of the data is within a target error bound and then reports the average. The data averaging period is variable. For a device that makes a daily measurement, the medical device may take weeks or even months before it reports a measurement, and may thereafter report daily updates to the trend based on an averaging period sufficient to ensure the reported measurement data is within a target error bound. It does not matter whether the average is based on an infinite impulse response filter or a finite response filter, in both cases the output variance of the filter is analyzed to ensure the data variation is within a specified bound. Additionally, the device can increase the filter bandwidth to allow error to approach the limit, thereby increasing the response time. The health reporting system can identify changes in trends and alter the averaging time to respond more quickly to a changing trend.

As an example a normal healthy individual begins making daily blood pressure measurements using the system. The system records the measures which have random errors of up to 25%. The system has a target accuracy of 3%. As the system receives new samples, the average value converges until the day-to-day fluctuation of the average is less than the target error. The system then reports the average as a data trend point. The system may also have a minimum number of samples required before it reports the trend data. The averaging may be a simple mean, or other low pass or band pass filter method. The averaging may employ, for example, a self-adjusting filter such as a Kalman type filter or employ outlier removal which may be associated, for example, with user error in making the measurement. If the error in the incoming data decreases, the system may decrease the averaging time to keep the variation in the filtered data close to the desired accuracy limit, versus averaging unnecessarily long. For example, the system may remove old samples from the average until the error increases to within a desired range of variance. For instance, if the user has relatively stable values for several weeks, the system will report a trend based on fewer number of more recent samples.

One problem addressed by the innovation is a change in trend. For instance, if the user becomes pregnant, goes on a diet, gets a chronic illness, or begins an exercise program such that the daily measured values increase in the variance, the system may increase the sample average period to obtain a more accurate reading. Alternatively, if the system has a long average and a new trend emerges, the data variance will increase, necessitating a longer filter average, just when the system needs speed the most. However, by monitoring the filtered data variation for different number of averages the system may identify that more filter bandwidth is needed to follow a rapid change. In this case increasing the filter bandwidth (decreasing the number of samples averaged) will allow the system to report data with the minimal response time within the target accuracy spec.

Additionally, the system may identify a trend from one or more trend data measurements, such as weight loss. The system may then remove the trend from the data prior to comparing the variance to the target error.

In some cases, a measurement that takes even a long time to make is better than no measurement, for instance if the measurement relates to a condition which if undiagnosed could become critical and for which clinical testing is infrequent, such as a colonoscopy for colon cancer. The mean time to death from colon cancer can be less than the mean period that adults get colonoscopies. If a measurement repeated conveniently (or automatically) at home took many months to report a result if would provide a revolutionary advance in medical treatment. From that perspective we invented a medical trending measurement system to ensure accurate data reporting by scaling the measurement time to ensure measurement error is within an established bound, such that measurements which were previously too inaccurate to meet a target medical standard can be used to record and report a data trend that does fall within a target error bound.

In an example the system is a health data collecting device having a medical measurement device that produces health data, a health data analyzer, a data error target, and a filtered data (trend) reporter. Without limitation, the health data collecting device may be a home appliance such as a toilet or weight scale, or a wearable device. The health data device measurement may include a random error and a systematic error and a drift error. The systematic error such as a measurement offset can be removed by appropriate calibration to within the target data error over the relevant measurement range. The drift error over the desired calibration period of the device should be smaller than the target data error. The random error, however, can be much larger than a target data error.

The health collecting device makes repeated measurements of a user health parameter such as, but not limited to heart rate, breathing rate, heart rate variability, resting heart rate, maximum heart rate, blood pressure, cardiac output, stroke volume, galvanic skin resistance, body impedance, body composition such as fat percentage, urine specific gravity, urine glucose, urine ketone bodies, pregnancy hormone, other hormones, drug or drug metabolite in urine, protein assay, stress markers, other urinalysis measures typically measured by color change chemistry or color change strip or electrochemical measurement, blood composition measurements, stool measurements, skin pigmentation or color, muscle tone, blood glucose, bone density, fingernail composition, breath volatile compound composition, blood oxygen saturation, lactate, breast milk composition, menstrual flow, fertility cycle, tumor size, wound healing, and so on.

In an example the system is a toilet with a urinalysis measurement system that measures several urine components including glucose, urea, creatinine, and uric acid and electrolytes such as Na and Cl. The system may report urea without any averaging, since it is present in large concentrations and the measurement error is small. However, glucose concentration is small, typically a fraction of a percent, and measurement error for typical concentrations may exceed a useful level. The system may average multiple measurements taken over a period of many days to obtain an average and report the average as trend data, where the variance of the trend data is less than a target error, and is a useful level of accuracy.

Over time the system may establish bounds of confidence on the measurement random error by estimating a width of an error distribution function, such as calculating a variance. Measurements well outside the normal range of the distribution function can be either flagged as an excursion for alerting a user or health care professional, or removed from the data by appropriate thresholding.

Based on the estimated errors of the averaged data the system can identify or report a statistically significant trend, such as weight loss or gain. The system can report statistically correlated trends, such as a toilet sensor that records excrement volume (amount of food eaten) and weight gain/loss to stress levels. The system can calculate a cumulative sum to identify when changes in trends occurred.

A health data reporting system which dynamically minimizes its averaging time to report trend (average) measurements where the trend variation is within a target error. A health data reporting system which takes data from multiple samples and reports averages of multiple samples, but not individual measurements. A health data reporting system which can identify and report statistically significant trends from dynamically averaged data. A health data reporting system which can report the variance expected in the averaged data. A health data reporting system which can flag or reject outliers by collecting a series of data, establishing a distribution width and comparing a difference of the data in question and the averaged data to a distribution width.

A key thing about this invention is that we do not release individual measurements, only validated averages. We prevent people and doctors from knee-jerk reacting to ups and downs in data they otherwise would not have had without the ubiquitous measurement system. It is a total revolution in the way of thinking as it pertains to health measurement reporting.

Other health data reporting systems may show averages or trends, but the difference here is the dynamic and independently optimized moving averaging duration which ensures the data incoming is within an optionally range-based accuracy target.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings, in which:

FIG. 1 is a perspective view of one embodiment of a toilet communicating to a network, cell phone and computer in accordance with an embodiment of the invention;

FIG. 2 is a front view showing an individual sitting on the toilet along with the health measuring devices communicating the measurements to the filter and the database in accordance with an embodiment of the invention;

FIG. 3 is an isometric view of a toilet showing various sensors and health measurement devices in accordance with an embodiment of the invention;

FIG. 4A is a chart showing the comparison of a referenced vs predicted values of Glucose concentration in a scatter plot from an example in accordance with an embodiment of the invention;

FIG. 4B is a chart showing the comparison of referenced vs predicted values of Glucose concentration of a normal user's urinalysis over time for an example in accordance with an embodiment of the invention;

FIG. 5A is a chart showing the comparison of referenced vs predicted values of Glucose concentration with a Kalman Filter for an example in accordance with an embodiment of the invention;

FIG. 5B is a chart showing the comparison of referenced vs predicted values of Glucose concentration with a Gaussian Filter for an example in accordance with an embodiment of the invention;

FIG. 6A is a chart showing the standard error (raw) predicted and filtered data for an example in accordance with an embodiment of the invention;

FIG. 6B shows the Gaussian filtered data for an example in accordance with an embodiment of the invention;

FIG. 7 is a flow chart showing the filtering process in accordance with an embodiment of the invention;

FIG. 8 is a flow chart detailing the recursive step sequences yielding the filtered output in accordance with an embodiment of the invention;

FIG. 9 is a flow chart showing how the predetermined target variance data inform the filtering function for each specific analyte sample being tracked in accordance with an embodiment of the invention.

### DETAILED DESCRIPTION OF THE DRAWINGS

It will be readily understood that the components of the present invention, as generally described and illustrated in the Figures herein, could be arranged and designed in a wide variety of different configurations. Thus, the following more detailed description of the embodiments of the invention, as represented in the Figures, is not intended to limit the scope of the invention, as claimed, but is merely representative of certain examples of presently contemplated embodiments in accordance with the invention. The presently described embodiments will be best understood by reference to the drawings, wherein like parts are designated by like numerals throughout.

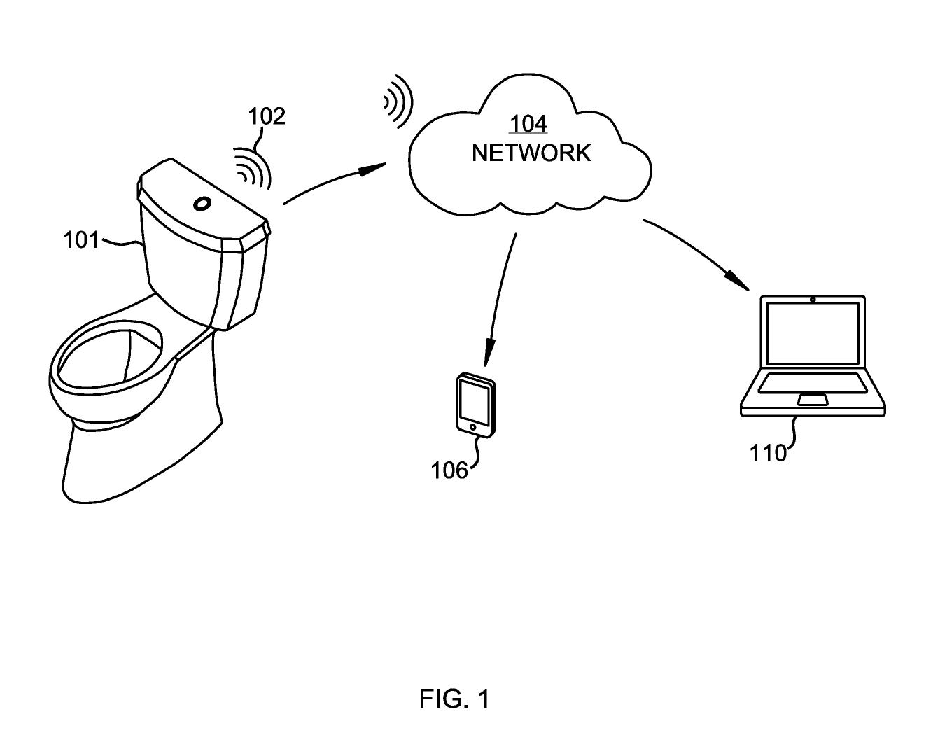

FIG. 1 is a perspective view of one embodiment of a toilet 101 communicating health data via a wireless signal 102 to a network database 104. This data is accessible via a cell phone 106 or a computer 110.

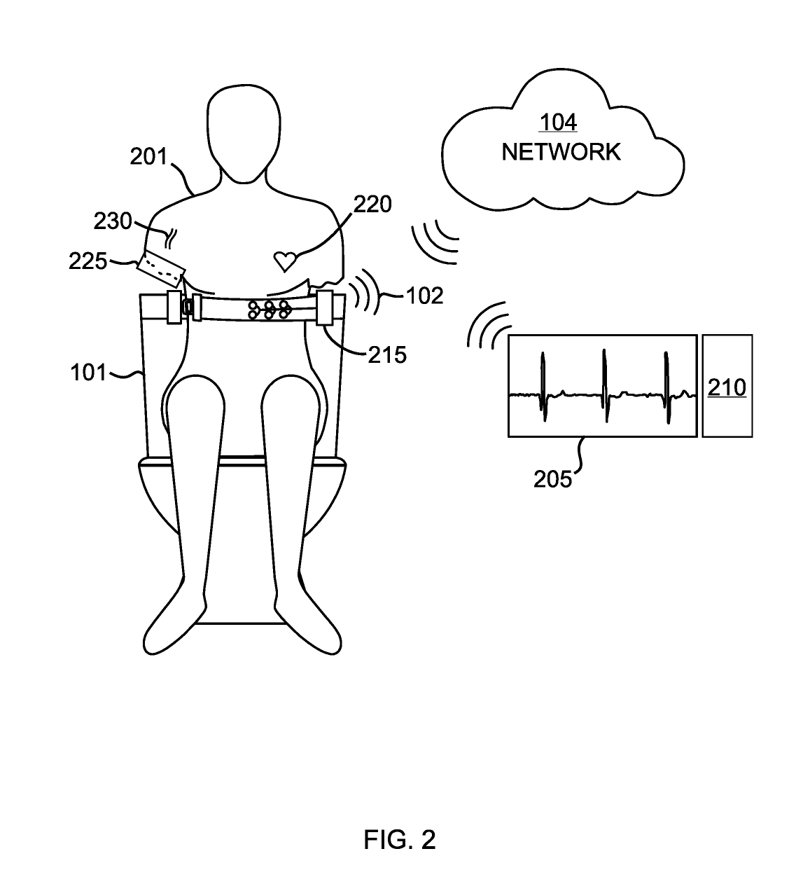

FIG. 2 is a front view showing an individual 201 during a health measurement session urinating or defecating in a toilet 101. A health measurement device 215 is shown gathering data and communicating the data via the wireless signal 102 to the filter 205, the processor 210 and the network database 104. The analyte sample of heart rate 220 is being measured by the measurement device 215. The analyte sample of blood pressure 230 is being measured by the measurement device 225.

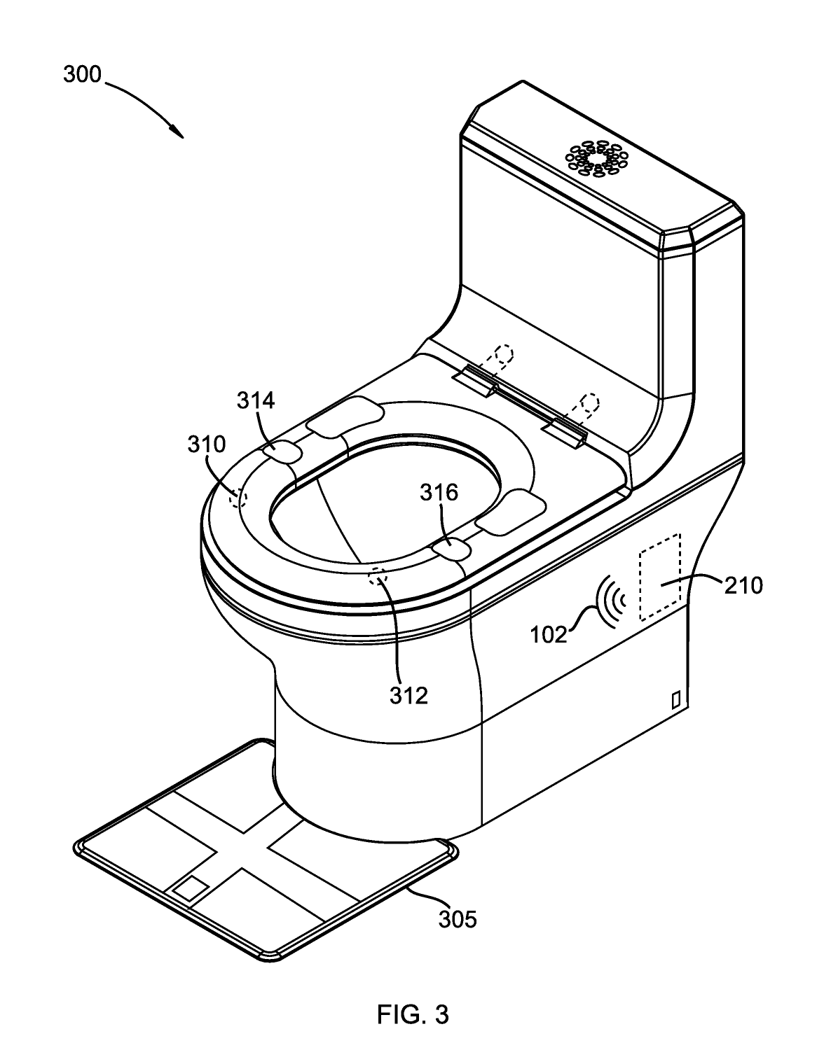

FIG. 3 is an isometric view of a toilet with multiple health measurement devices shown. All of the health measurement devices communicate the collected data to the processor 210. A wireless signal 102 communicates data to the network or from adjunct devices. A scale 305 is shown along with pressure sensors 310 and 312 collect weight measurements. bioimpedance sensors 314 and 316 are in contact with the individual's skin to collect multiple analyte samples.

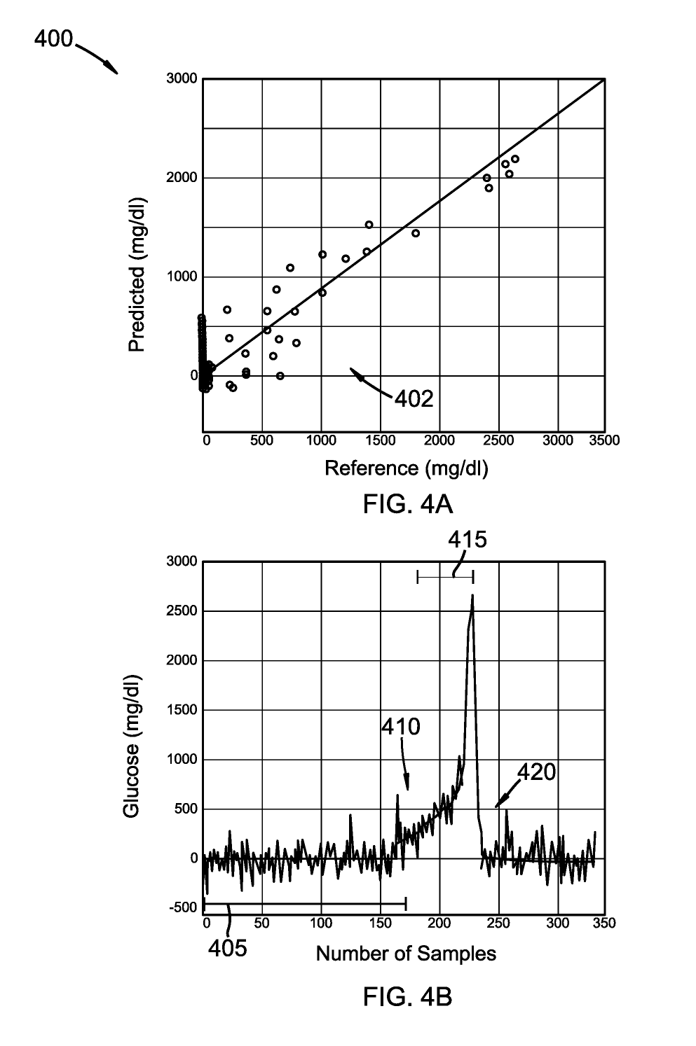

FIG. 4B shows a smaller spread in data at high concentrations, with clinically relevant accuracy. However, at low concentrations, time sequence filtering is needed to improve the signal to noise (SNR) ratio. This chart compares predicted glucose to a reference measurement from the commercial AU480 color change chemistry-based analysis instrument. An illustration of how the filtering function is carried out is described in the following example: Over three hundred samples 402 were validated with the calibration model. Most of the samples are at low glucose level, in the normal range (˜30 mg/dl). Our research is mainly focused on detecting the changes in glucose concentration of normal users, and observing any significant increase over time. The error at the low concentration is larger than is reported in previous studies, because the compact spectrometer used in this study is not high-end scientific grade instrument, and our toilet-deployable setup was exposed to external variables such as ambient light, slight temperature variation, or evaporation of a small amount of a sample in the urine capture slot. Since the error window is large compared to the normal range, it is hard to predict if the user's glucose level is in the normal level. Therefore, digital filters were employed to remove noise by filtering the predicted data from a sequence of user samples collected over many days. Each measurement corresponds to a unique sample. The error is dominated by measurement noise. That is, if we repeat the spectral measurement on a single sample, we obtain error on the order of what we see from measuring samples on different days. This means that in practice, the averaging could be done by measuring a single sample repeatedly. However, the purpose of this study is to demonstrate the efficacy of data filtering a sample sequence to obtain a trended result within a specified error bound. FIG. 4A shows a comparison of glucose concentration for reference to predicted data to hypothesize a normal user's urinalysis over time.

FIG. 4B shows a data series simulating the effect of a user's glucose level changing over time. The reference data were arranged in order of ascending glucose concentration and the data series cut and spliced to generate four regions of interest: normal glucose concentration 405, (2) a step in glucose concentration 410, (3) a ramp, (4) a return to normal. (It is not possible to cause a person to generate samples with this behavior.) The smooth line is the measured (actual) glucose level. The erratic line is the predicted glucose level obtained from multivariate analysis of the urine transmission spectrum. The glucose concentration increases at about 170^{th} sample and dramatically increased after that, which may be similar to the situation in which a medicated user stops medication. After the 280th sample the glucose level decreases back to the normal range, which may be typical of a user beginning an effective therapy. For high levels of glucose, the spectral signature of glucose rises out of the spectral noise and the accuracy of the prediction improves.

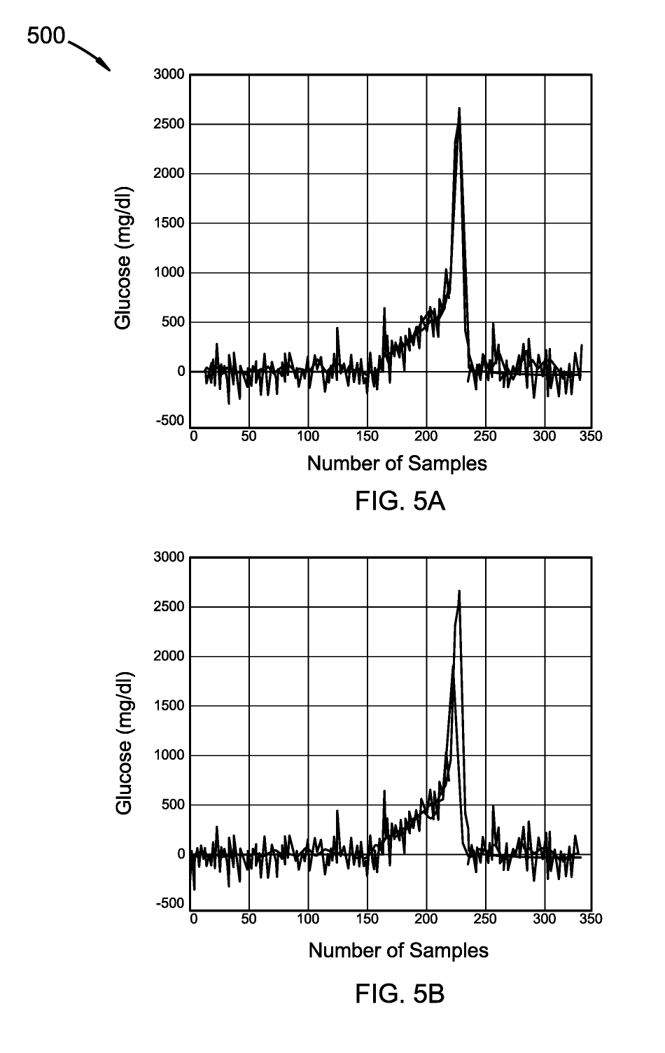

FIG. 5A shows a comparison of the reference and the predicted glucose to the filtered data showing a Kalman filter. Filtered data are shown as the small deviation erratic line, and root mean squared error of prediction and standard deviation is shown in Table 1.

TABLE 1
RMSEP and RMSEP2

| | Predicted data | Kalman | Gaussian |
|---|---|---|---|
| RMSEP | 133.45 | 88.43 | 113.60 |
| RMSEP2 | 94.04 | 28.18 | 32.00 |
| STD | 121.60 | 30.37 | 30.60 |

RMSEP was calculated by

$$RMSEP = \sqrt{\frac{\sum {( {\overline{y}}_{i} - {y}_{i , ref} )}^{2}}{N}} , i = 1 , … , N \qquad ( 1 )$$

N, y̅ᵢ, and y_{i,ref} represent the total number of samples, the predicted glucose value, and the reference glucose value, respectively. RMSEP2 in Table 1 represents RMSEP selecting only reference data under 20 mg/dl, which is the normal concentration of urine glucose. In comparison of the raw predicted data to filtered data, RMSEP of filtered data are lower than that of the raw data. Notably RMSEP2 is 66% lower than the unfiltered data. STD in Table 1 represents standard deviation of the first 150 samples at low glucose level. STD of predicted data were 4× smaller than unfiltered data. For comparison, simple averaging of 20 samples with random error produces SNR improved of \sqrt{20}≅4.5.

The individual measurement has low accuracy, however, in terms of tracking and trending for a user with a large set of data, filters can reduce the measurement noise and the predict glucose levels with much less error and disclose the accurate glucose trend. By looking at the STD of the filtered data, we have an estimate of the error of the filtered (trended) data. This opens the opportunity to tune the filter, as needed, to obtain a trended measurement within a target error bound. For instance, if the STD is too large, the window size can be increased. In this scheme, the individual sample measurements are not trusted results and are not reported. This is a big change from the way data has been collected and analyzed in the health care field, where individual measurements have to be trusted because trended measurements are not generally available. By reversing the assumption, namely that a series of data are available for trending but accuracy is unknown, data filtering and analysis of the filtered data variance (or similarly STD) can provide a measurement with (1) bounded confidence and (2) resilience to potential outliers. Outliers may come from sample variation, instrument error, environment effects or sample handling. The downside of trended data is that averaging reduces the time resolution. One solution is to use an adaptive filtering scheme that attempts to minimize the combination of errors from measurement and from a changing sample trend. For the extreme situations considered in FIG. 3, a simple Kalman filter was sufficient to obtain sufficiently accuracy to categorize glucose as in the normal range, adapt to step changes, and follow sharp rises and falls.

FIG. 5B shows a comparison of the reference and the predicted glucose to the filtered data showing a Gaussian filter with window size of 20, which represents about 3-4 days of toilet use for a typical user. Window size determines the width of the smoothing window.

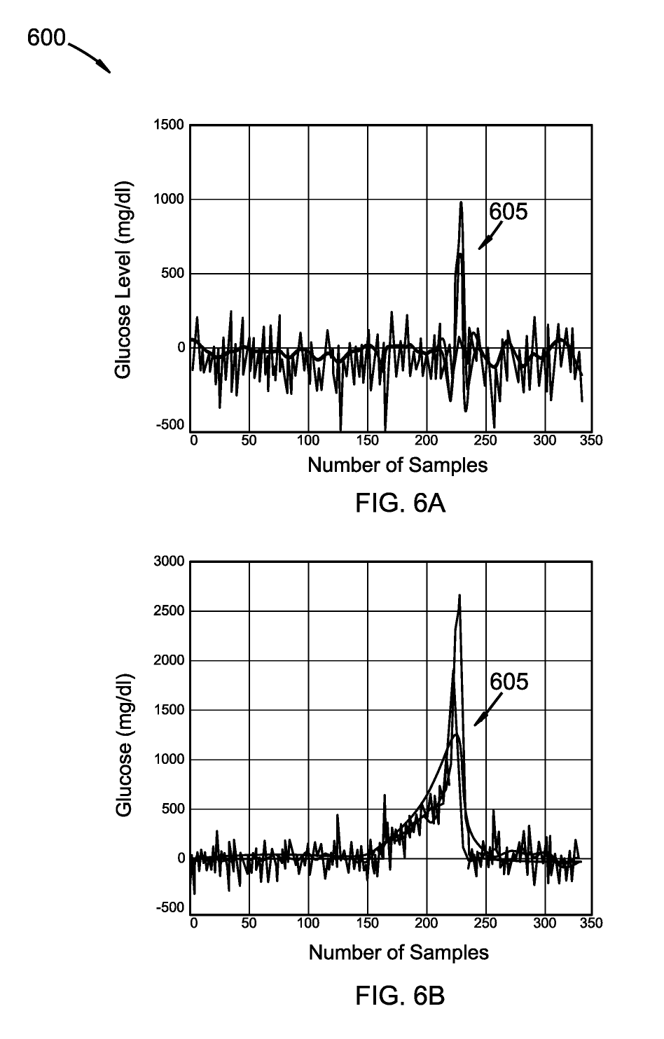

FIG. 6A shows the standard error of (raw) predicted and filtered data. The largest error occurs at the high peak 605, which is due to the finite filter bandwidth producing a filtered result that lags the rapidly changing data.

FIG. 6B shows the Gaussian filtered data with different window sizes. The window size five can easily follow the increase but it is still noisy at the low concentration. In contrast, the window size fifty smoothed out the overall data and cannot follow up the increase. It might be hard to meet both at the same time. Window size also can be determined with a standard deviation. By varying window size to meet the target standard deviation, the desired accuracy can be achieved. The proper filters and optimal tunings for the filter parameters will be dependent on the interest and the application of research. Adaptive filters can be tuned to increase the bandwidth to follow sudden increases and provide a narrower bandwidth (more smoothing or larger window) for data that is approximately steady.

NIR spectroscopy enables in-toilet urinalysis, providing more timely information than colorimetric assay urine tests from medical labs, which provide doctors with only an infrequent snapshot of a patient's medical condition. However, compact NIR instrumentation used for this study and others lacks sensitivity and stability to detect normal low levels of urine glucose. However, by averaging data from different urine samples over time, an accurate glucose level trend is obtained. This scheme for averaging many samples to achieve improved SNR, is a totally different approach from traditional diagnoses that rely on presumed accurate single measurements. Significantly, the variance of the filtered data can be monitored and the filter bandwidth adjusted such that the trended result meets a desired level of accuracy, even when the individual sample measurements cannot. This radical scheme enables remote medical measurements to provide trended health data for preventative care and unobtrusive patient monitoring, and is especially useful for health trends that change slowly. Notably, the filter performance can be optimized to provide sufficient averaging to achieve a target accuracy level and still follow sharp trends in the data. The trended measurements of multiple samples over time may provide a better overall picture of a user's medical progress than isolated lab samples that are not robust against outlier data. Medical diagnostics using trended data with validated and tunable accuracy can expand the role of health tracking and disease management to a new level of usefulness and cost efficacy.

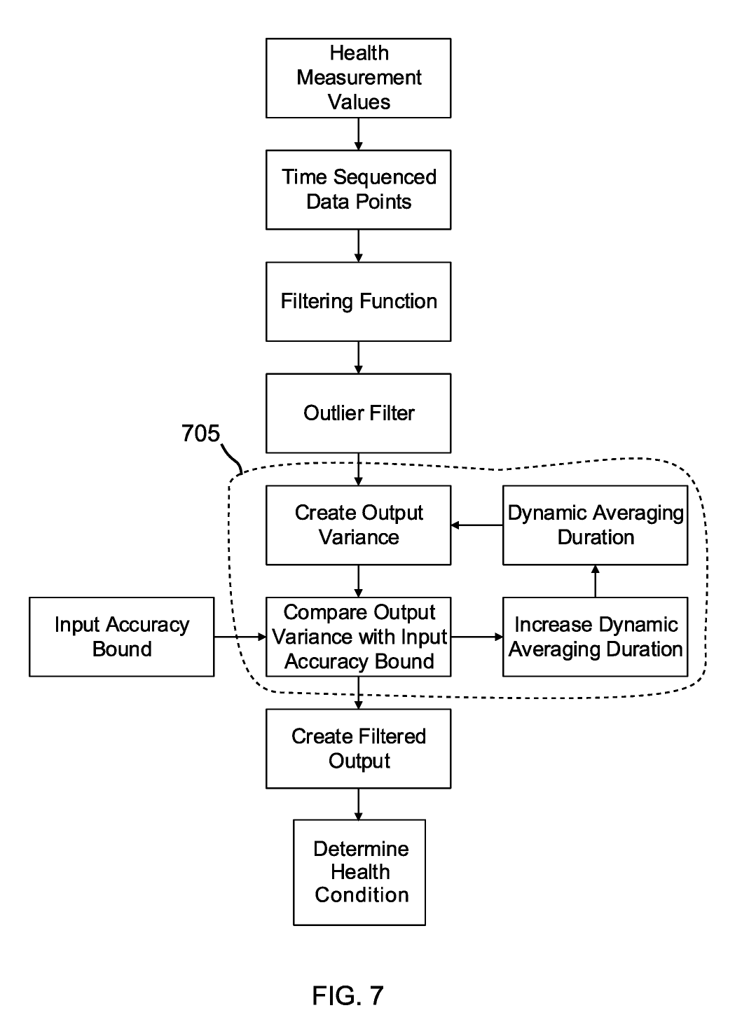

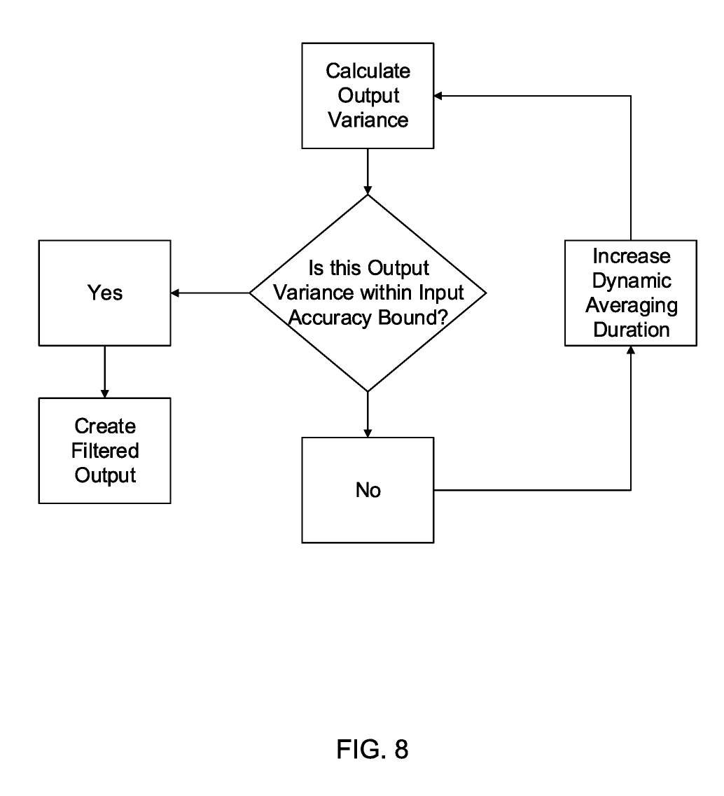

The flow chart in FIG. 7 shows the overall flow of data through the filtering process. Inputs include health measurement values and input accuracy bound. The output is the determination of a health condition. The decision 705 on whether to continue filtering is further described in FIG. 8

FIG. 8 is a flow chart that shows the detail of how the decision of whether to continue filtering or to provide the filtered output is done. The reiterative loop continues to compare the collected analyte sample data to the previous series of samples until the values are within the input accuracy bound. Once the accuracy is met, the filtered output is the determination of a health condition.

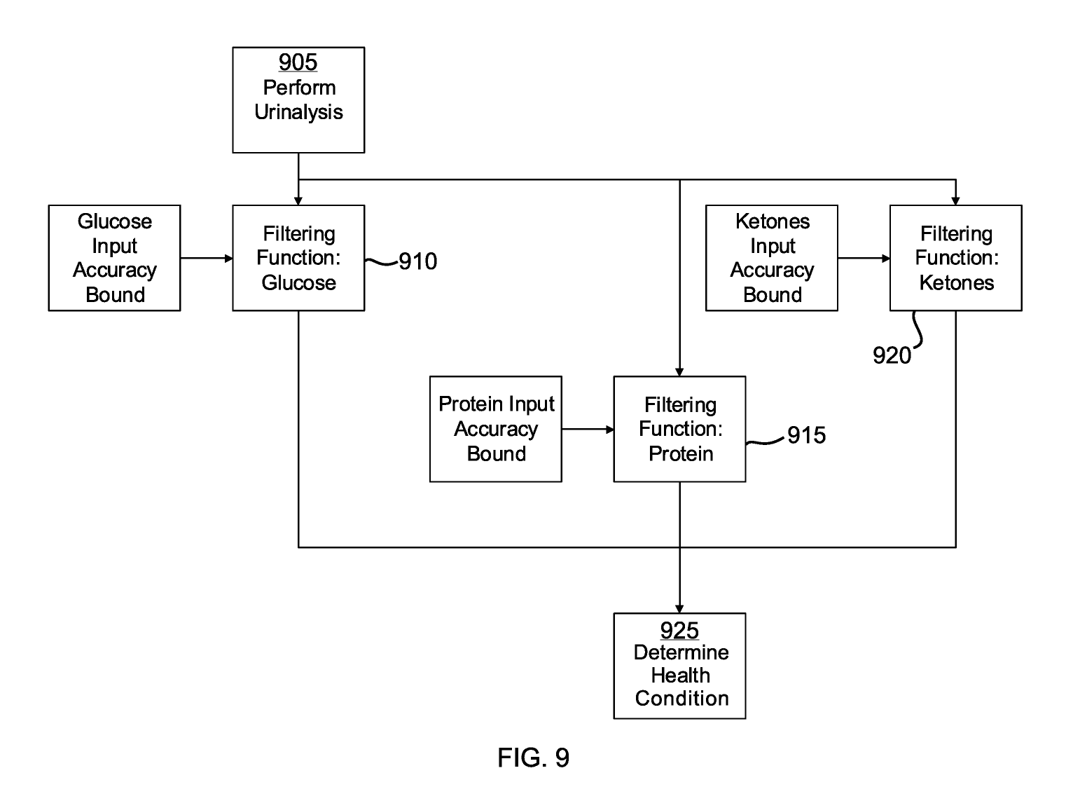

FIG. 9 is a flow chart that demonstrates how data from a single process—in this example urinalysis 905, is used to perform multiple analyses on the components of glucose 910, protein 915 and ketones 920. Each of these components influence each other, and enhance the refinement of the analyte sample. The values (or levels) of each of these components is used in the filtering process to determine a new value (or values) for the next step of the reiterative process. For example, glucose levels and ketones inform the protein filtering process allowing a more accurate protein report to be passed on for the next iteration of the protein filtering function. Once all of these values are within the prescribed accuracy bound, a health condition is determined based on these values 925.

1. A health condition determination method, comprising:
providing an analyte sample;
providing a predetermined target variance for one or more properties of the analyte sample;
providing a toilet comprising one or more health measurement devices in communication with the analyte sample;
providing multiple toilet-based health measurement sessions gathering data related to the analyte sample using the one or more health measurement devices;
providing one or more processors connected to the one or more health measurement devices, the one or more processors receiving the data and executing a filtering function for refining the data;
using the processors to track the filtered data over the multiple sessions;
using the filtered data recursively to refine a measured variance for the one or more properties of the analyte sample until a refined measured variance is within the predetermined target variance;
using the one or more properties of the analyte sample when the predetermined target variance is reached to determine a health condition of a donor who provided the analyte sample.

2. The health condition determination method of claim 1, wherein the analyte sample is a sample within a range of normal health conditions.

3. The health condition determination method of claim 1, wherein the analyte sample is a sample outside a range of normal health conditions.

4. The predetermined target variance of claim 1, wherein the target variance is a specific predefined variance based on the analyte sample being sampled.

5. The health condition determination method of claim 1, wherein the target variance is changed based on a modified standard deviation which is revised according to the filtered data after each toilet-based health measurement session specific to an individual.

6. The health condition determination method of claim 1, wherein the one or more health measurement devices that is in communication with the analyte sample by means of a sensor is selected from one or more of: a light based sensor, a sound sensor, a color sensor, a digital sensor, an analog sensor, a spectrometer, a refractometer, a camera based particle sizer, an x-ray, a doppler sensor, an infrared sensor, a monochromator, a sonogram sensor, a magnetic resonance imaging sensor, a cardiograph sensor, an electrocardiograph sensor, an echocardiogram sensor, a scale, a pressure sensor, a thermometer, a temperature sensor, a glucose monitor, an interferometer, a colorimeter, a stethoscope, a glucose polarization analyzer, an infrared spectroscopy device, an absorption detector, a reflectance detector, a transmission detector, a conductivity sensor, a polarographic flow analyzer, an oxygen electrode, a body fat measuring apparatus, a current or voltage electrode, a blood pressure measuring apparatus, a camera, a microscope, a particle size analyzer, an optical detector, a proximity sensor, an ultrasonic sensor, a flow sensor, a chemoreceptor, a biosensor, bioimpedance sensor, or an oximetry device.

7. The health condition determination method of claim 1, wherein the one or more health measurement devices in communication with the analyte sample is an adjunct to the toilet.

8. The health condition determination method of claim 7, wherein the adjunct to the toilet is a urine hat.

9. The health condition determination method of claim 7, wherein the toilet-based health measurement sessions comprise urinating or defecating in an adjunct to the toilet.

10. The health condition determination method of claim 1, wherein the analyte sample is one or more of: glucose, blood pressure, red and white blood cells, heart rate, lung sounds, body temperature, cholesterol, bone marrow, antigens, cancer, minerals, vitamins, nutrients, proteins, hormones, hCG levels, body weight, toxic substances, drugs, waste substances, urea, or lactic acid.

11. The health condition determination method of claim 1, wherein the health condition information is stored in a medical database accessible by an individual or an individual's chosen medical personnel via a computer or phone with an internet connection.

12. The medical database accessible by an individual or the individual's chosen medical personnel of claim 11, wherein the health condition information is only recorded, stored and is accessible locally in a local database.

13. The health condition determination method of claim 1, wherein the health condition information is stored in a national medical database.

14. The health condition determination method of claim 1, wherein the tracking occurs over an extended period of time.

15. The tracking of claim 14, wherein the extended period of time includes daily reporting for at least one day up to 30 days.

16. The tracking of claim 14, wherein the extended period of time includes daily reporting for at least one day up to 90 days.

17. The tracking of claim 14, wherein the extended period of time includes daily reporting for at least one day up to one year or the lifetime of the individual.

18. The health condition determination method of claim 1, wherein the toilet-based health measurement sessions comprise urinating or defecating in the toilet.

19. The toilet-based health measurement sessions of claim 18, wherein the data gathered relating to the analyte sample occurs every time the toilet is used.

20. The health condition determination method of claim 1, wherein the health data measurements are taken while a person is using the toilet.
